(19) United States
(12) Patent Application Publication
(10) Pub. No.: US 2008/0091039 A1
(43) Pub. Date: Apr. 17, 2008

(54) Hydrogenation Process and High Monoene Compositions Obtained Therefrom

(75) Inventor: Ronald T. SLEETER, Decatur, IL (US)

Correspondence Address: STERNE, KESSLER, GOLDSTEIN & FOX P.L.L.C., 1100 NEW YORK AVENUE, N.W., WASHINGTON, DC, 20005, US

(73) Assignee: Archer-Daniels-Midland Company, Decatur, IL (US)

(21) Appl. No.: 11/871,747

(22) Filed: Oct. 12, 2007

Related U.S. Application Data
(60) Provisional application No. 60/851,289, filed on Oct. 13, 2006.

(51) Int. Cl. C07C 51/36

(52) U.S. Cl. 554/145; 554/1

(57) ABSTRACT

A hydrogenation process for producing high monoene compositions is disclosed. In particular, a catalyst comprising basic copper carbonate is used for the selective hydrogenation of oils that contain unsaturated fatty acyl components such as unsaturated vegetable oils. Hydrogenation according to the methods of the invention yield high monoene compositions that are useful for industrial applications, such as lubricants.

### BACKGROUND OF THE INVENTION

1. Field of the Invention

The invention is directed to methods of hydrogenating unsaturated compositions and the hydrogenated products obtained therefrom.

2. Background Art

Bio-based oils, such as vegetable oils, represent a useful alternative to petroleum-based oils for use in industrial applications, such as lubricants. However, low cost commodity bio-based oils, such as commodity soybean oil, in their natural state are high in polyunsaturated fatty acids, which typically renders them oxidatively unstable. As many industrial oils require a certain degree of oxidative stability, commodity bio-based oils have had limited utility for such uses.

For example, soybean oil contains five different fatty acids (in the form of fatty acid acylglycerol esters) as its major components. These five fatty acids are: palmitic acid (C16:0) which averages about 11 percent by weight; stearic acid (C18:0) which averages about 4 percent by weight; oleic acid (C18:1) which averages about 20 percent by weight; linoleic acid (C18:2) which averages about 57 percent by weight; and linolenic acid (C18:3) which averages about 8 percent by weight of the total fatty acids.

The relative order of sensitivity to oxidation is linolenic>linoleic>oleic>saturates. The stability problem of soybean oil has been attributed to the oxidation of its fatty acids, and particularly to the oxidation of the linolenic acid (C18:3) component. This causes the oil to oxidize rapidly. Vegetable oil stability can be enhanced if the linolenic acid concentration can be reduced.

Since commodity soybean oil currently marketed today contains relatively high amounts of linolenic acid (7-10%) compared to other vegetable oils such as corn oil which has about 1%, its use is often constricted unless the linolenic content is reduced. Typically, the linolenic acid content should be below 1-2% in order to have the widest application and to qualify for rigorous industrial use environments such as for lubricants, metal cutting fluids, and transformer oils.

To address the problems of soybean oil due to the linolenic acid content, various processing approaches have been proposed. Such processing of the soybean vegetable oil includes: (1) minimizing the ability of the fatty acids to undergo oxidation by adding metal chelating agents, antioxidants, or packaging in the absence of oxygen; or (2) the elimination of the endogenous linolenic acid by selective hydrogenation. These approaches have not been entirely satisfactory. The additional processing is expensive, time consuming, commonly ineffective, and frequently generates undesirable by-products.

Hydrogenation (a chemical reduction by means of adding hydrogen across a double bond) of unsaturated substrates such as unsaturated oils can be used to increase oxidative stability and thus improve performance attributes by lowering the amount of linolenic, linoleic, and oleic acids in the oils. Hydrogenation is widely used for obtaining products which can be used in various fields, from the food industry to the field of plastic materials and the like. Thus, polyunsaturated oils are hydrogenated to reduce the degree of unsaturation in the oil, prior to subsequent processing to obtain secondary products, such as food grade oils, additives, lubricants and the like.

Reduction of the double bond content in polyunsaturated oils is traditionally carried out by partial hydrogenation using gaseous hydrogen, in the presence of a suitable catalyst. Hydrogenation conditions to reduce the oxidatively unstable species in vegetable oil, such as polyunsaturated acids linolenic and linoleic acid, are being studied by many in the industry. Those catalysts currently being examined are generally precious metal based, and hydrogenation is carried out under extremely mild conditions, such as low temperatures. However, to date this has generally resulted in increased saturated fatty acid content and/or the use of very expensive catalysts.

Catalysts vary in degree of selectivity. The selectivity referred to in this context is the ability of preferentially reducing linolenic acid before linoleic acid and oleic acid. Selectivity in this context also applies to the ability of a catalyst to reduce by hydrogenating only to form monoenes, without reducing to full saturation.

Many current industrial methods of hydrogenation use catalysts which are not selective. Such catalysts form substantial amounts of saturated fatty acids, which increases the melting points of the resultant products. Oils high in saturated fatty acids are stable but are typically solid at room temperature, rendering such oils unsuitable for many industrial applications. Many industrial oils, such as lubricants, must be liquid at a wide range of temperatures.

Precious metal catalysts are generally the most active and also the least selective. They typically produce high amounts of saturated fatty acids for a minimal reduction of linolenic acid. Furthermore, precious metal catalysts can be poisoned from various minor components in oils. As a result, activity is lost over time and reaction conditions must be continually monitored and altered. These catalysts may be employed in column reactors which require emptying and recharging after the useful catalyst life has ended. The catalyst then must be returned to the catalyst company for credit and regeneration. All of this involves catalyst loss and added cost for column recycling. As precious metal catalysts lose activity and must be recovered, users of precious metal catalysts are often required to purchase a large excess of precious metal to form a “pool” or “kitty” of precious metal, so that the catalyst producer can provide fresh catalyst as needed. As a result, the use of precious metal catalysts is accompanied by a very large capital investment in precious metals.

Nickel catalysts are more selective and have a greater preference for reducing linolenic acid to monoene while producing less saturates. Nickel catalyzed hydrogenation uses small amounts of catalyst for relatively short periods of time to reduce the linolenic acid content to the desired range, which is often 1.5%. The oil may then additionally be winterized (chilled and cold filtered) to remove any crystalline fractions. However, the use of nickel catalysts still causes the content of saturated fatty acids to rise, leading to increasing levels of solid content as the content of saturated fatty acids increases. Thus, oil hydrogenated with nickel catalysts may lose much of the liquid character desired for industrial applications.

Copper-chromium combination catalysts (i.e., copper chromite catalyst) have hitherto been found to be the most selective for production of the monoene. The hydrogenation of the polyunsaturated oils with copper chromite can produce the corresponding monoene, with little or no production of saturated fatty acid.

However, copper chromite has low catalytic activity and requires very long reaction times. Also, the chromium must be recycled and disposed in a satisfactory manner. First, the catalyst must be recovered from the oil after the hydrogenation reaction by suitable means, such as by centrifugation or filtering. Traces of catalyst remaining in the oil must be removed in a thorough manner, such as filtering through bleaching earth. This removal generates significant quantities of solid waste containing spent copper chromite catalyst and would require shipment to a land fill or to a possible reclamation facility. In addition, the finely powdered catalyst containing chromium could pose a significant health risk to workers operating the processes.

Filtered oil further requires washing with a suitable solution of chelating agent to further recover chromium. This wash water requires passage through expensive ion exchange resin columns to reduce the chromium concentration in the water prior to discharge in order to achieve allowable limits. Further, regulatory permits to allow discharge of trace levels of chromium in waste water must be obtained. In order to measure chromium released to the environment, expensive analytical monitoring equipment and trained operators would be required. Based on the drawbacks of copper chromite-based catalyst, its commercial use as a hydrogenation catalyst has been limited.

Other non-chromium copper-based catalysts known in the art still have the disadvantage of being no faster than copper chromite in reaction time. Furthermore, some were fabricated on a support, generally a molecular sieve, making them somewhat expensive to make. In addition, high hydrogenation temperatures were required (170 to 200° C.). To prepare these catalysts, a support material was slurried in a solution of copper (II) nitrate, and sodium carbonate was added to precipitate copper (II) carbonate onto the support. This preparation was then heated to 350° C. for two hours.

Genetic varieties of soybeans containing oil with low linolenic acid levels have just begun to be commercialized. The most recent variety to be commercialized has utilized a traditional genetic breeding program for its development. In general, oils produced from genetic varieties are expensive alternatives to hydrogenated oils.

A high monoene oil would be useful for many lubricant applications where oxidative stability is needed along with good cold flow properties. For many applications, genetically modified high oleic soybean oil may be useful as a biodegradable renewable resource vegetable oil-based lubricant. The most widely accepted uses for genetically modified high oleic soybean oil are for applications formulated with additive packages to improve various performance parameters for use in environmentally sensitive locations. This would include motor boat engines, chain saws and irrigation pump oils, to name a few. However, high oleic soybean oil is relatively expensive and its availability is limited. Thus, high oleic soybean oil has not found much use.

It would be desirable to improve the oxidative stability of commodity bio-based oils to increase their utility as industrial oils, at a cost low enough to compete with petroleum-based oils. Thus, there is a continuing need in the fats and oils industry for an economical processes for hydrogenation of unsaturated compositions to selectively reduce unsaturated content, particularly linolenic acid without substantially increasing the saturated fatty acid content, while avoiding the disposal problems associated with use of chromium-containing catalysts.

### BRIEF SUMMARY OF THE INVENTION

An embodiment is directed to a process for hydrogenating a composition containing at least two sites of saturation. The process comprises contacting a first composition containing at least two sites of unsaturation with a hydrogenation catalyst comprising basic copper carbonate in a hydrogen atmosphere under conditions effective to form a hydrogenated composition. The hydrogenated composition has a ratio of monounsaturated content to saturated content of greater than about 2. The saturated content of the hydrogenated composition is not substantially higher than the saturated content of the first composition.

The inventive hydrogenation process uses a hydrogenation catalyst containing basic copper carbonate to selectively hydrogenate vegetable oils to a high monoene content without substantially increasing the saturated content, using minimal amounts of catalyst with minimal activity loss. Thus, high monoene oils can be prepared from unsaturated compositions such as commodity bio-based oils using the process of the invention for industrial oil applications such as lubricants, metalworking fluids, transformer fluids, cosmetics, inks, releasing agents, wetting agents, dispersing agents, viscosity control agents, crystallization agents, softening agents, emollients, anti-dusting agents, nutritional ingredients, and personal care products.

### DETAILED DESCRIPTION OF THE INVENTION

In an embodiment, the invention is directed to a hydrogenation process. The process comprises contacting a composition containing at least two sites of unsaturation with a hydrogenation catalyst comprising basic copper carbonate in a hydrogen atmosphere under conditions effective to form a hydrogenated composition having an increased or elevated monounsaturated content, without a substantial increase in saturated content over the starting composition.

### Suitable Compositions for Hydrogenation

Suitable compositions for the hydrogenation process include any composition containing at least two sites of unsaturation. Such compositions can comprise a single compound or mixtures of compounds wherein at least one compound contains at least two sites of unsaturation.

Polyunsaturated fatty acyl compositions comprise compounds and mixtures that contain compounds of the following generic structure:

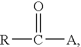

wherein R is a carbon chain from about 2 to about 23 carbons and contains at least two sites of unsaturation; A can be a residue of a monohydric alcohol, a diol, polyol, or glycerol, or a hydroxy, alkoxy or aryloxy moiety. The above general structure includes the following substructure:

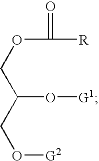

wherein R is as described above, and G¹ and G² are each independently selected from the group consisting of hydrogen and,

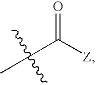

wherein Z represents a carbon chain from about 2 to about 23 carbons in length, optionally having at least two sites of unsaturation. This formula encompasses the fatty acid esters commonly found in vegetable oils and polyunsaturated vegetable oils such as palmitic acid (C16:0 or 16:0); stearic acid (C18:0 or 18:0); oleic acid (cis C18:1 or cis 18:1); linoleic acid (cis C18:2 or cis 18:2); and linolenic acid (cis C18:3 or cis 18:3).

Preferably, the fatty acyl composition containing at least two sites of unsaturation is a vegetable, animal or synthetic fat or oil, or derivatives or mixtures thereof. References made herein to “fatty acids” are intended to mean fatty acids in the form of fatty acid esters in the fatty acyl composition, that is a vegetable, animal or synthetic fat or oil, or derivatives or mixtures thereof, unless the fatty acid is specifically referred to as a “free fatty acid.” In this context, it is preferred that the fatty acid or derivative thereof is a triglyceride, diglyceride or monoglyceride or alkyl ester containing a residue of the fatty acid.

References to levels of “fatty acids” in oils refer to the level of fatty acid chains in the form of esters such as glycerides. For example, a fatty acyl containing composition comprising one or more polyunsaturated (i.e. two or more sites of unsaturation) vegetable fatty acid(s) or derivatives or mixtures thereof can include the fatty acids contained in oils in the form of fatty acid esters.

In the generic structure above, when A is a residue of glycerol, then the fatty acyl composition can comprise a triglyceride, diglyceride and/or monoglyceride of a fatty acid (i.e., glycerol alkanoates), and mixtures thereof. Such a diglyceride or triglyceride will have two or three fatty acid chains, respectively, wherein at least one of the chains has at least two sites of unsaturation. More preferred mono-, di- and triglycerides include glycerides of vegetable oil fatty acids. Most preferably, such glycerides are naturally occurring in a vegetable oil starting material.

In the generic structure above, A can be a residue other than glycerol, such as a monohydroxyl alcohol or a polyhydroxy alcohol. The monohydroxyl alcohols or the polyhydroxyl alcohols can be primary, secondary or tertiary alcohols of annular, straight or branched chain compounds. The monohydroxyl alcohols may comprise methyl alcohol, isopropyl alcohol, allyl alcohol, ethanol, propanol, n-butanol, iso-butanol, sec-butanol, tert-butanol, n-pentanol, iso-pentanol, n-hexanol, hexadecyl alcohol or octadecyl alcohol. Polyhydroxy alcohols may comprise bioderived polyols suitable for use according to various embodiments of the present disclosure comprising diols, such as glycols comprising ethylene glycol and propylene glycol; saccharides, such as, but not limited to biobased polyols including monosaccharides include dioses, such as glycolaldehyde; trioses, such as glyceraldehyde and dihydroxyacetone; tetroese, such as erythrose and threose; aldo-pentoses such as arabinose, lyxose, ribose, deoxyribose, xylose; keto-pentoses, such as ribulose and xylulose; aldo-hexoses such as allose, altrose, galactose, glucose (dextrose), gulose, idose, mannose, talose; keto-hexoses, such as fructose, psicose, sorbose, tagatose; heptoses, such as mannoheptulose and sedoheptulose; octoses, such as octolose and 2-keto-3-deoxy-manno-octonate; and nonoses, such as sialose; disaccharides; oligosaccharides, such as raffinose(melitose), stachycose, and verbascose; sorbitol, glycerol, sorbitan, isosorbide, hydroxymethyl furfural, polyglycerols, plant fiber hydrolyzates, fermentation products from plant fiber hydrolyzates, and various mixtures of any thereof.

In this preferred embodiment, the fatty acyl containing composition is an animal or vegetable oil. Preferred oils include vegetable oils. Suitable vegetable oils include but are not limited to: soybean oil, linseed oil, sunflower oil, canola oil, rapeseed oil, cottonseed oil, peanut oil, safflower oil, derivatives and conjugated derivatives of said oils, and mixtures thereof. These oils are known as polyunsaturated vegetable oils. Most preferably, the oil is soybean oil.

### Hydrogenation Process

The process described herein is useful for fully hydrogenating or partially hydrogenating a composition. As such, the terms “hydrogenation” or “hydrogenating” as used herein are intended to include partial hydrogenation.

The hydrogenation process includes contacting the starting composition with a hydrogenation catalyst comprising basic copper carbonate, and heating the mixture under a hydrogen atmosphere for a time suitable to provide a hydrogenated composition having desired properties. The desired properties of the hydrogenated composition include an increased or elevated monounsaturated content, particularly without a substantial increase in the saturated content compared to the starting composition.

The temperature for which the starting composition and hydrogenation catalyst are heated can be any temperature(s) from about 50° C. to about 250° C. For example, the temperature is from about 100° C. to about 250° C., or from about 100° C. to about 200° C., or from about 160° C. to about 200° C., or from about 140° C. to about 220° C., or any range within the above-listed ranges. Illustratively the temperature is about 160° C., about 180° C., or about 200° C.

The term “hydrogen atmosphere” refers to the atmosphere in contact with the unsaturated composition as comprising hydrogen gas. The pressure of hydrogen in the process can be in the range of about 5 psi to about 1000 psi. For example, the pressure can be from about 20 psi to about 150 psi, or from about 40 psi to about 80 psi, or any range within the above ranges. Suitably, the pressure is about 60 psi.

The time for which the mixture is heated under a hydrogen atmosphere is dependent, inter alia, upon the catalyst used and the desired properties of the resulting hydrogenated composition. For example, the time can range from about 1 minute to about 48 hours (for example, about 30 minutes to about 8 hours, or about 30 minutes to about 7 hours), or any range in between.

Longer reaction times tend to produce hydrogenated compositions having higher monoene content and thus are particularly useful for industrial applications. Particularly useful reaction times range from about 4 hours to about 7 hours (for example, about 4 hours, about 5 hours, about 6 hours, or about 7 hours).

### Hydrogenation Catalyst

The hydrogenation catalyst used in the hydrogenation process of the invention comprises copper carbonate/copper hydroxide, also known in the art as basic copper carbonate or malachite, having chemical formula Cu₂(OH)₂CO₃. The terms “copper carbonate/copper hydroxide,” “basic copper carbonate,” and “malachite” are used interchangeably herein unless otherwise indicated.

Basic copper carbonate is a product of commerce, typically containing about 50+ % copper carbonate, with the remainder typically containing copper hydroxide. Suitable basic copper carbonate material can be obtained from World Metal, LLC (Sugar Land, Tex., USA). The density of the material can range from about 500 to about 2000 kg/cubic meter. The material is basic in character and insoluble in water. As received from the manufacturer, the material can be green in color. However, supplies often vary in shades of color and density (darker green or olive, and heavier, lighter or fluffier) reflecting variations in raw materials and manufacturing procedures. Despite variations in the physical appearance of the material, the amount of contained copper metal remains essentially constant.

The basic copper carbonate may also be in the form of natural malachite mineral. Natural malachite can be found in the oxidation zone of polymetallic deposits in ore fields, and appear as radiate-fibrous, spheroidal, and sintered aggregates with shell-like cleavage, silky luster, and a characteristic green color in varicolored band due to diverse grain sizes. Natural malachite can be purchased in clumps from rock collectors, and may contain trace amounts of phosphorus, calcium, strontium, zinc and manganese.

Basic copper carbonate can also be synthetically prepared by any suitable method. For example, basic copper carbonate can be “precipitated basic copper carbonate,” i.e., basic copper carbonate prepared by a precipitation method. Illustratively, basic copper carbonate can be prepared by precipitation of copper cations and carbonate anions. A suitable method of preparation of basic copper carbonate is disclosed in H. Parekh and A. Hsu, “Preparation of synthetic malachite. Reaction between cupric sulfate and sodium carbonate solutions,” Industrial & Engineering Chemistry Product Research and Development 7(3): 222-6 (1968). A precipitated basic copper carbonate catalyst is also described in Example 4, below.

The basic copper carbonate used in the hydrogenation catalyst can be chemically treated, i.e. treated with a chemical reagent. For example, the basic copper carbonate can be treated with a hydrogen peroxide solution. Illustratively, chemically treated basic copper carbonate can be prepared by contacting basic copper carbonate with a hydrogen peroxide solution while maintaining the mixture at temperatures from about −5° C. to about 100° C. (for example, from about 5° C. to about 30° C.); and separating the solid material from the mixture.

The hydrogen peroxide used to chemically treat the basic copper carbonate can be in the form of an aqueous solution. Concentrations of aqueous hydrogen peroxide can range from about 1% to about 90% hydrogen peroxide. For example, the concentration is from about 40% to about 60%, or from about 45% to about 55%. Illustratively, the concentration is about 50% as supplied commercially.

The hydrogen peroxide-treated basic copper carbonate may be subjected to one or more additional chemical treatments with a hydrogen peroxide solution. Prior to additional chemical treatment(s), the basic copper carbonate may be rinsed, filtered, and/or dried.

The preparation of the hydrogenation catalyst can also include disrupting agglomerates or clumps in the material. Agglomerates or clumps in the catalyst material can be disrupted before and/or after the solid material is separated from the mixture.

The agglomerates or clumps in the catalyst material can be disrupted by any suitable method. For example, the material can be tumbled, deagglomerated, ground, stirred, or slurried (with or without grinding).

Illustratively, agglomerates or clumps in the catalyst material are disrupted by grinding, preferably by slurry grinding in an appropriate liquid. For example, the material can be slurry ground in hydrogen peroxide, which can be the same or different from, and at the same or different concentration of, the hydrogen peroxide that may be optionally used to chemically treat the basic copper carbonate. After slurry grinding, the catalyst can be separated from the liquid phase by any method known in the art such as filtering (e.g. vacuum filtering), decanting, centrifuging, or any combination thereof. Optionally, the material can then be dried by vacuum, heating or other drying method known in the art.

In embodiments of the invention, the catalyst (comprising basic copper carbonate, including as commercially available, natural malachite, and/or synthetically-prepared basic copper carbonate) used in the hydrogenation process is an unsupported catalyst. For example, an unsupported catalyst comprising basic copper carbonate can be used for hydrogenation for a composition containing at least two sites of unsaturation to obtain hydrogenated compositions as disclosed herein.

The hydrogenation catalyst can be further treated prior to use in a hydrogenation reaction in order to improve its ability to catalyze a hydrogenation reaction. For example, the catalyst can be heated in an oil in the presence or absence of additional hydrogen. The further treated catalyst is then recovered from the oil and can be used to catalyze hydrogenation reactions as disclosed herein.

The oil that can be used in the further treatment of the hydrogenation catalyst is not particularly limited, and can include any vegetable oil, animal oil, butterfat, cocoa butter, cocoa butter substitutes, illipe fat, kokum butter, milk fat, mowrah fat, phulwara butter, sal fat, shea fat, borneo tallow, lard, lanolin, beef tallow, mutton tallow, tallow, animal fat, canola oil, castor oil, coconut oil, coriander oil, corn oil, cottonseed oil, hazelnut oil, hempseed oil, jatropha oil, linseed oil, mango kernel oil, meadowfoam oil, mustard oil, neat's foot oil, olive oil, palm oil, palm kernel oil, peanut oil, rapeseed oil, rice bran oil, safflower oil, sasanqua oil, shea butter, soybean oil, sunflower seed oil, tall oil, tsubaki oil, tung oil, marine oils, menhaden oil, candlefish oil, cod-liver oil, orange roughy oil, pile herd oil, sardine oil, whale oils, herring oils, triglyceride, diglyceride, monoglyceride, triolein palm olein, palm stearin, palm kernel olein, palm kernel stearin, triglycerides of medium chain fatty acids, and derivatives, conjugated derivatives, genetically-modified derivatives and mixtures thereof.

The temperature and time for which the catalyst/oil mixture is heated to further treat the catalyst is not particularly limited. For example, the temperature can be from about 100° C. to about 200° C., and the time ranges from about 1 minute to about 120 minutes, typically about 15 minutes.

In an embodiment, the hydrogenation catalyst contains basic copper carbonate as the only active catalytic material. Basic copper carbonate catalyst is robust enough to remain active for the period of time necessary to catalyze hydrogenation of compositions to elevated monoene levels. However, the hydrogenation catalyst can optionally include one or more other catalytically active materials, provided that such materials can be used in tandem with the basic copper carbonate to produce a hydrogenated composition having an elevated monoene content without substantially increasing the saturated content from the starting composition. The hydrogenation catalyst preferably does not include copper chromite due to the drawbacks as discussed above for this material.

The hydrogenation catalyst can be reused or recycled. For example, after the starting composition has been hydrogenated, the solid material comprising the hydrogenation catalyst is separated from the hydrogenated oil.

Separation of the solid material from a hydrogenated oil can be performed by any suitable methods, including centrifugation, settling, decantation, filtration (e.g., vacuum filtration), contact with a filter aid, contact with a liquid or solid chelating agent, addition of an activated adsorbent, or any combination thereof. For example, vacuum filtration can be carried out using filter aids, such as Celite 503 Diatomaceous Earth (World Minerals Inc., Goleta, Calif.). Other separation methods include contact with a liquid or solid chelating agent such as citric acid solution, by addition of activated adsorbent such as activated SorbsilR92 (INEOS Silicas Americas, LLC, Joliet, Ill.), and filtering through a filter aid.

Subsequent to separation, the solid material can reused as the hydrogenation catalyst with the process as disclosed herein. This process can be repeated such that the solid material comprising the hydrogenation catalyst is used to hydrogenate a second or additional composition(s), then separated from the resulting hydrogenated compositions for reuse.

### Hydrogenated Compositions

In another embodiment of the invention, hydrogenated compositions prepared by the inventive process have increased or elevated monounsaturated content, and preferably without a substantial increase in saturated content, compared to the starting compositions.

By “increased” or “elevated” monounsaturated content, it is meant that the monoene content of the hydrogenated composition is higher than that of the starting composition. The monounsaturated content of a composition can be calculated by methods known in the art.

For example, commodity soybean oil typically contains 20 percent by weight oleic acid (C18:1). However, the amount of oleic acid can vary depending on the soybean or other oil used as the starting material. The examples below illustrate use of a refined bleached and deodorized soybean oil having a monoene content of 27% as a starting composition.

The monounsaturated content of the hydrogenated compositions can be increased to about 40% by weight or higher, for example, about 45%, or about 50%, about 55%, about 60%, about 65%, about 70%, or higher, for example 75%, 80%, or higher. Any range of monounsaturated content between these values is contemplated, e.g. about 40% to about 75%, about 45% to about 85%, or about 45% to about 70%. The upper limit of monounsaturated content is typically limited by the amount of saturated content present in the hydrogenated composition.

For example, the monounsaturated fatty acid content of a polyunsaturated vegetable oil, such as e.g. soybean oil can be increased, for example, to about 40% or higher by weight or higher, for example, about 45%, or about 50%, about 55%, about 60%, about 65%, about 70%, or higher by weight. If the starting soybean oil contains palmitic acid (C16:0) in the amount of about 11 percent by weight and stearic acid (C18:0) which averages about 4 percent by weight, the oleic acid content can be increased up to about 85% using the inventive hydrogenation process.

Hydrogenated compositions of the invention typically have a ratio of monounsaturated content to saturated content of greater than about 2 (i.e., about 2 or higher), suitably about 3 or higher. For example, the ratio of monounsaturated content to saturated content can be from about 2 to about 6, for example, about 3 to about 5, or about 3 to about 4.5, or about 3.25 to about 4.25, including ranges in between these ranges.

The saturated content of the hydrogenated composition (for example, a hydrogenated vegetable oil such as soybean oil) is not substantially higher than the saturated content of the starting composition. By “a saturated content not substantially higher than the saturated content of the first [or starting] composition,” it is meant that the amount of saturated content does not substantially increase during the hydrogenation process. For example, the saturated content of the hydrogenated composition does not increase by more than about 5 percentage points by weight, preferably not by more than about 2 percentage points by weight, more preferably not by more than about 1.5 percentage points by weight, even more preferably not by more than about 1 percentage point by weight, more preferably by not more than about 0.5 percentage points by weight, and most preferably by not more than about 0.1 percentage point by weight. For example, the saturated content of the hydrogenated composition may be substantially unchanged from the first or starting composition (e.g., within about 0.5 percentage points by weight).

In an embodiment, oils prepared by the inventive hydrogenation process have a ratio of 18:1 to 18:0 of greater than 6.0 and a content of 18:0 which is substantially unchanged from the content of 18:0 originally present in the starting oil.

In another embodiment, oils made by the inventive hydrogenation process have a ratio of 18:1 to 18:0 of greater than 6.0 and a content of saturated fatty acids which is substantially unchanged from the content of saturated fatty acids originally present in the starting oil.

In yet another embodiment, oils made by the inventive hydrogenation process have a ratio of cis 18:1 to 18:0 of greater than 6.0 and a content of 18:0 which is substantially unchanged from the content of 18:0 originally present in the starting oil.

In still yet another embodiment, oils made by the inventive hydrogenation process can have a ratio of cis 18:1 to 18:0 of greater than 6.0 and a content of saturated fatty acids which is substantially unchanged from the content of saturated fatty acids originally present in the starting oil.

In still yet another embodiment, oils made by the inventive hydrogenation process can have a ratio of cis 18:2 to 18:0 of greater than 0.05 and less than 11 and a content of 18:0 which is substantially unchanged from the content of 18:0 originally present in the starting oil.

In still yet another embodiment, oils made by the inventive hydrogenation process can have a ratio of 18:2 to 18:0 of greater than 0.05 and less than 11 and a content of saturated fatty acids which is substantially unchanged from the content of saturated fatty acids originally present in the starting oil.

In still yet another embodiment, oils made by the inventive hydrogenation process contain a content of 18:0 which is substantially unchanged from the content of 18:0 originally present in the starting oil and one or more of a ratio of 18:1 to 18:0 of greater than about 6, a ratio of cis 18:1 to 18:0 of greater than 6, a ratio of 18:2 to 18:0 of greater than about 0.2 and less than about 11.6, a ratio of cis 18:2 to 18:0 of greater than about 0.1 and less than about 11.1.

In still yet another embodiment, oils made by the inventive hydrogenation process have a content of saturated fatty acids which is substantially unchanged from the content of saturated fatty acids originally present in the starting oil and one or more of a ratio of 18:1 to 18:0 of greater than about 6, a ratio of cis 18:1 to 18:0 of greater than 6, a ratio of 18:2 to 18:0 of greater than about 0.2 and less than about 11.6, a ratio of cis 18:2 to 18:0 of greater than about 0.1 and less than about 11.1.

Using the inventive hydrogenation process described herein, hydrogenated vegetable oils containing the following ratios of fatty acids can be prepared:

Illustrative Oil 1) C18:1/C18:0 above about 6.0; C18:2/C18:0 above about 6.0; and C18:0 no greater than about 5%;

Illustrative Oil 2) C18:1/C18:0 above about 10.0; C18:2/C18:0 above about 3.2; and having C18:0 no greater than about 5%;

Illustrative Oil 3) C18:1/C18:0 above about 13.9; C18:2/C18:0 above about 3.9; and having C18:0 no greater than about 5%;

Illustrative Oil 4) C18:1/C18:0 above about 10.7; C18:2/C18:0 above about 7.2; and C18:0 no greater than about 5%;

Illustrative Oil 5) C18:1 cis/C18:0 above about 6.6; C18:2/C18:0 above about 7.2; and C18:0 no greater than about 5%.

The degree of hydrogenation of the compositions of the inventions can be measured by the Iodine Value, which can be determined according to AOCS method Cd 1c-85. Hydrogenated compositions of the invention have an Iodine Value of less than about 120; for example, about 70 to about 120, about 70 to about 105, or suitably about 85 to about 105.

The invention can be used to prepare oils low in linolenic acid and lower in saturated fatty acids than partially hydrogenated oils prepared by conventional processes, such as with nickel catalysts. The hydrogenated oils of the invention have good oxidative stability due to the lowered content of linolenic acid and remain a fluid composition due to the low content in saturated fatty acids such as stearic acid (18:0) and palmitic acid (16:0). Thus, the hydrogenation process produces vegetable oils having a high content of monoenoic fatty acids, such as oleic acid and elaedic acid (collectively referred to as 18:1), that are not substantially enriched in saturated fatty acids.

### Applications for Hydrogenated Compositions of the Invention

The inventive process yields hydrogenated compositions that are particularly useful for industrial applications. Illustrative applications for use of these compositions include, but are not limited to lubricants, metalworking fluids, transformer fluids, cosmetics, inks, releasing agents, wetting agents, dispersing agents, viscosity control agents, crystallization agents, softening agents, emollients, anti-dusting agents, nutritional ingredients, and personal care products.

In addition, the hydrogenated oils can be used as a starting material for derived processes and products, such as feedstock for lipid modifications such as fractionation and chemical or enzymatic transesterification or interesterification reactions to prepare useful triacylglycerols, diacylgycerols, monoacylglycerols, esters and waxes. The hydrogenated oils can also be blended with other oils or fats to provide a blend having desired characteristics.

Derivatives of these oils include genetically modified oils. One desired trait of genetically modified oils is the lower content of linolenic acid compared to natural oils. Some low level varieties have linolenic acid levels as low as about 1.2 to about 1.6%. In natural varieties, the level of linolenic acid is generally about 7-10%. Low linolenic acid varieties can benefit substantially by the hydrogenation method of the invention especially when the level of linolenic acid is above about 2%, but below the usual amount contained in the corresponding natural variety.

When applied to a vegetable oil, the present method of hydrogenation advantageously yields a hydrogenated or partially hydrogenated vegetable oil with desirable characteristics for use where liquid oils are needed, such as in lubricants, metalworking fluids, transformer fluids, cosmetics, inks, releasing agents, wetting agents, dispersing agents, viscosity control agents, crystallization agents, softening agents, emollients, anti-dusting agents, nutritional ingredients, and personal care products.

Hydrogenated compositions of the invention can have properties that make them particularly suitable for lubricant applications. For example, hydrogenated compositions of the invention can be used in motor oil applications, and are expected to meet requirements set forth for motor oil. Some requirements and data generated for motor oil are as follows:

TABLE 1
Motor Oil Base Oil Properties

| Gasoline Engine Oil | | |
|---|---|---|
| Specs. | NOACK Volatility | Viscosity Index (VI) |
| High Oleic Soy* | 1 | 220 |
| Commodity Soy* | 0.002 | 213 |
| PAO 4 | 12 | 120 |
| Group III | 15 | 120-145 |
| Group II | 23 | 95-115 |
| Group I | 29 | 95-100 |

PAO = Poly alpha olefins
*Reported analytical results (from Valvoline Co., Proceedings of the United Soybean Board Soy Lubricants Technical Advisory Panel, Sep. 16, 2003, “Update on Soy Program at Valvoline” by Dr. Frances Lockwood). These terms in the table are used as defined therein.

Regulations from governmental legislation have forced lubricant companies to move to higher standards, moving to PAO 4 standards. Thus, there is increased need to have lower NOACK volatility and higher VI. Motor oils can be blended with various amounts of high oleic soybean oil to achieve these higher standards. High oleic soy oil also has improved oxidative stability over commodity soy oil and has improved tolerance to the extremes of typical engine operation. Thus, compositions of the invention having high monoene content can be used as a motor oil or as part of a motor oil blend.

Compositions of the invention can also be used as a blended component of hydraulic fluid. Some testing was reported using various additive packages to high oleic soybean oil (Glancy, “High Oleic Soybean Oil in Hydraulic Fluids” in Proceedings of the United Soybean Board Lubricants and Fluids Technical Advisory Panel Meeting, Sep. 13, 1999). The results for a 10% proprietary additive package tested after a period of 1000 hours of use was reported as follows:

| | | |
|---|---|---|
| Viscosity at 100° C. | 9.8 | cSt |
| Acid Number | 7.5 | mg. KOH/gram |
| Four Ball Wear | 0.35 | mm scar. |
| Oxidative Stability | 20 | (ASTM D2272) |

The conclusion was that high oleic soybean oil displayed excellent performance as a lubricant base stock without additives and had excellent oxidative stability when formulated with a commercial antioxidant package. Thus, high monoene compositions of the invention can be used as industrial hydraulic fluid, incidental food contact hydraulic fluid, incidental food contact gear oil, chain bar fluid and for grease applications.

Hydrogenated compositions of the invention may contain additives that are desirable for a particular industrial oil application. For example, the hydrogenated compositions may also contain one or more of antioxidants, detergents, dispersants, metal deactivators, antiwear agents, extreme-pressure agents, viscosity index modifiers, pour point depressants, foam inhibitors, demulsifiers, friction-modifiers, and/or corrosion-inhibitors.

Fatty acid ratios as described herein may be derived by determining the fatty acid profile of starting oils and hydrogenated oil by gas chromatography (GC) according to AOCS methods. Reactions may also be monitored by refractive index (RI).

Oxidative stability relates to how easily components of an oil oxidize, and can be measured by instrumental analysis such as Oil Stability Index, Accelerated Oxygen Method (AOM), and Pressure Differential Scanning Calorimetry. The degree of oxidative stability (AOM) is rated as the number of hours to reach a peroxide value of 100.

In all aspects of the invention, the temperature, temperatures or ranges represent the temperature at which a step is conducted. However, the temperature can be more than one temperature in the given range because of fluctuations in temperature during the step.

### EXAMPLES

### Example 1

Commercial basic copper carbonate previously purchased from Mallinckrodt Laboratory Chemicals (Phillipsburg, N.J.) (1.03 grams) was used as a catalyst to hydrogenate refined bleached and deodorized (RBD) soybean oil having a monoene content of 27%, palmitic acid content of 10.45, and a stearic acid content of 4.66%. The hydrogenation reaction was carried out in a pressure reactor at 200° C. and 60 psi hydrogen for 7 hours with a slight flow of hydrogen added during the reaction. A hydrogenated oil having a monoene content of 64.3% with no substantial increase in stearic acid, palmitic acid, or total saturated fatty acid content was produced.

### Example 2

Hydrogenation of refined bleached and deodorized soybean oil was repeated substantially as in Example 1 with the same catalyst and conditions. The hydrogenation produced a hydrogenated oil having a monoene content of 54.4% in 7 hours with no substantial increase in stearic acid, palmitic acid, or total saturated fatty acid content.

### Example 3

A hydrogenation reaction was conducted substantially under the same conditions as in Example 1, except that the basic copper carbonate was hydrogen peroxide-activated. Basic copper carbonate previously purchased from Mallinckrodt Laboratory Chemicals was slurried in water and cooled on ice. Hydrogen peroxide (50%, 1 ml per gram of the basic copper carbonate) was added slowly over a period of one hour to the water slurry of the basic copper carbonate with cooling over ice. The catalyst was filtered, dried and ground slightly prior to use.

1.03 grams of the hydrogen peroxide-activated basic copper carbonate catalyst was used to hydrogenate 600 grams of RBD soybean oil for seven hours. A hydrogenated oil was produced having 47.8% monoene with no substantial increase in stearic acid content, palmitic acid, or total saturated fatty acid content.

### Example 4

Unsupported precipitated malachite was prepared in accordance with the description in H. Parekh and A. Hsu, “The Preparation of Malachite. Reaction between cupric sulfate and sodium carbonate solutions,” Industrial & Engineering Chemistry Product Research and Development 7(3): 222-6 (1968), incorporated by reference herein in its entirety. Basic copper carbonate (10.15 grams) from World Metals, Inc. (Sugar Land, Tex.) was slurried in 50 ml water. Concentrated sulfuric acid (6.6 ml) was added, followed by an additional 20 ml of water to make a copper sulfate solution. Sodium carbonate (12 g) was dissolved in 200 ml water to make a sodium carbonate solution. The first 66% of the sodium carbonate solution was added dropwise at room temperature to the copper sulfate solution. The last 34% was added by pouring in slowly. A precipitate formed, which was recovered by decanting and siphoning off the supernatant. The precipitate was washed with water and dried in a vacuum oven to yield a basic copper carbonate catalyst which was used as a hydrogenation catalyst without further modification.

The catalyst (1.0188 grams) was used in a hydrogenation reaction with 600 grams of refined, bleached, deodorized soybean oil containing 27% monoene. The reaction temperature set point was 200° C. and the reactor was charged with 60 psi hydrogen gas. A slight flow of hydrogen was added during the reaction. The total monoene content was increased to 67.0% after seven hours reaction time with no substantial increase in stearic acid, palmitic acid, or total saturated fatty acid content.

### Example 5

A commercially available basic copper carbonate from World Metals Inc. was used as received as a hydrogenation catalyst substantially as in Example 1 (1.0257 grams of catalyst to 600 grams of oil). The particle size distribution of the basic copper carbonate was 18% greater than 20 microns; 47 percent between 11.5 and 20 microns; 4.5% between 8 and 11.5 microns; and the remainder less than 8 microns. After 7 hours of reaction under the conditions of Example 1, a hydrogenated oil having a monoene content of 54.4% with no substantial increase in stearic acid, palmitic acid, or total saturated fatty acid content was obtained.

### Example 6

A commercially available sample of basic copper carbonate (CUCOCER) from World Metals was dissolved in water and treated with a 50% solution of hydrogen peroxide, filtered, dried and ground substantially as in Example 3. This catalyst was used in a hydrogenation reaction substantially under the conditions as in Example 1. The monoene content of the oil increased to 42% at 4 hours, 45.5% at 5 hours, and 49.4% at 6 hours, with no substantial increase in stearic acid, palmitic acid, or total saturated fatty acid content.

### Example 7

A commercially available nickel based catalyst was run for comparison. Thus 0.6166 grams of G96B from Sud Chemie Company (Louisville, Ky. 40232) was added to 670 grams of soybean oil at 200° C. with a hydrogen pressure of 25 psi. This produced a hydrogenated oil having a monoene content of 62.3% in 4 hours, but increased the saturated fatty acid content to 35.2% from about 16.5% in the starting material.

The fatty acid composition of oils hydrogenated with the catalysts of the invention (Examples 1-6) and a nickel catalyst control (Example 7) is provided in Table 2, below. Small amounts of other fatty acids that are present are not shown in Table 2.

The ratios of fatty acids in a) the starting soybean oil, and b) oil obtained after hydrogenating soybean oil according to the methods above (referenced below by Example No.) were calculated and are provided in Table 3, below.

TABLE 2
Fatty acid composition of oils hydrogenated with basic copper carbonate catalysts (Exs. 1-6)
and a nickel catalyst control (Ex. 7)

| | | | | 18:1 | Total | | 18:2 | Total | | 18:3 | Total | Total | Iodine |
|---|---|---|---|---|---|---|---|---|---|---|---|---|---|
| Example | 16:0 | 18:0 | 18:1 cis | trans | 18:1 | 18:2 cis | trans | 18:2 | 18:3 cis | trans | 18:3 | trans | Value** |
| SBO* | 10.45 | 4.7 | 26.9 | | 26.9 | 52.9 | | 52.9 | 7.2 | | 7.2 | 0.20 | 133.6 |
| 1 | 10.52 | 4.6 | 35.5 | 28.8 | 64.3 | 13.9 | 4.5 | 18.4 | 0 | 0.09 | 0.09 | 33.4 | 87.4 |
| 2 | 10.42 | 4.8 | 33.0 | 21.4 | 54.4 | 23.1 | 4.6 | 27.7 | 0.02 | 0.21 | 0.23 | 26.2 | 95.4 |
| 3 | 10.46 | 4.7 | 31.3 | 16.5 | 47.8 | 29.3 | 4.8 | 34.1 | 0.06 | 0.27 | 0.33 | 21.5 | 101.0 |
| 4 | 10.47 | 4.8 | 36.6 | 30.4 | 67.0 | 9.7 | 5.8 | 15.5 | 0.02 | 0.15 | 0.17 | 36.4 | 84.9 |
| 5 | 10.49 | 4.7 | 32.0 | 18.1 | 50.1 | 25.1 | 6.9 | 32.0 | 0.12 | 0.44 | 0.56 | 25.5 | 100.0 |
| 6*** | 10.66 | 4.6 | 32.3 | 17.3 | 49.4 | 27.8 | 5.7 | 33.5 | 0.04 | 0.20 | 0.24 | 23.3 | 101.3 |
| 7 | 10.49 | 23.6 | 22.5 | 39.9 | 62.4 | 0.66 | 1.7 | 2.36 | 0 | 0 | 0.00 | 41.6 | 57.8 |

*Starting soybean oil for all reactions
**Iodine values calculated according to AOCS method Cd 1c-85; Triglyceride Iodine value Calculation.
***Values after 6 hours of hydrogenation

TABLE 3
Ratios of fatty acids in oils of the Examples

| | | | | | Monoun- |
|---|---|---|---|---|---|
| Descrip- | | | | Cis | saturates/ |
| tion | 18:1/18:0 | 18:2/18:0 | Cis18:1/18:0 | 18:2/18:0 | Saturates |
| SBO* | 5.72 | 11.26 | 5.70 | 11.23 | 1.78 |
| Example 1 | 13.98 | 4.00 | 7.72 | 3.02 | 4.25 |
| Example 2 | 11.33 | 5.77 | 6.88 | 4.81 | 3.57 |
| Example 3 | 10.17 | 7.26 | 6.66 | 6.23 | 3.15 |
| Example 4 | 13.96 | 3.23 | 7.63 | 2.02 | 4.39 |
| Example 5 | 10.66 | 6.81 | 6.81 | 5.34 | 3.30 |
| Example 6 | 10.78 | 7.28 | 7.02 | 6.04 | 3.25 |
| Example 7 | 2.64 | 0.10 | 0.95 | 0.03 | 1.83 |

Having now fully described this invention, it will be understood to those of ordinary skill in the art that the same can be performed within a wide and equivalent range of conditions, formulations, and other parameters without affecting the scope of the invention or any embodiment thereof.

All documents, e.g., scientific publications, patents, patent applications and patent publications, if cited herein are hereby incorporated by reference in their entirety to the same extent as if each individual document was specifically and individually indicated to be incorporated by reference in its entirety. Where the document cited only provides the first page of the document, the entire document is intended, including the remaining pages of the document.

1. A hydrogenation process comprising:
contacting a first composition containing at least two sites of unsaturation with a hydrogenation catalyst comprising basic copper carbonate in a hydrogen atmosphere under conditions effective to form a hydrogenated composition;
said hydrogenated composition having a ratio of monounsaturated content to saturated content of greater than about 2, and a saturated content not substantially higher than the saturated content of the first composition.

2. The process of claim 1, wherein hydrogenation occurs at a temperature of about 50° C. to about 250° C.

3. The process of claim 1, wherein hydrogenation occurs at a pressure of about 40 psi to about 80 psi.

4. The process of claim 1, wherein hydrogenation occurs for a time period of about 4 hours to about 8 hours.

5. The process of claim 1, wherein the catalyst comprises hydrogen peroxide-treated basic copper carbonate.

6. The process of claim 1, wherein the catalyst comprises a natural malachite mineral.

7. The process of claim 1, wherein the catalyst comprises precipitated basic copper carbonate.

8. The process of claim 1, wherein the catalyst is unsupported.

9. The process of claim 1, wherein said first composition comprises a fatty acyl-containing composition, wherein said fatty acyl contains at least two sites of unsaturation in a carbon chain.

10. The process of claim 9, wherein said fatty acyl-containing composition comprises a polyunsaturated vegetable, animal or synthetic fat or oil, or derivatives or mixtures thereof.

11. The process of claim 10, wherein said oil is a vegetable oil.

12. The process of claim 11, wherein said vegetable oil is selected from the group consisting of soybean oil, linseed oil, sunflower oil, canola oil, rapeseed oil, cottonseed oil, peanut oil, safflower oil, derivatives and conjugated derivatives of said oils, and mixtures thereof.

13. The process of claim 1, wherein said hydrogenated composition is a vegetable oil comprised of fatty acid chains having a profile selected from the group consisting of:
(a) C18:1/C18:0 above about 6.0; C18:2/C18:0 above about 6.0; and
C 18:0 no greater than about 5%;
(b) C18:1/C18:0 above about 10.0; C18:2/C18:0 above about 3.2; and
having C18:0 no greater than about 5%;
(c) C18:1/C18:0 above about 13.9; C18:2/C18:0 above about 3.9; and
having C18:0 no greater than about 5%; and
(d) C18:1/C18:0 above about 10.7; C18:2/C18:0 above about 7.2; and
having C18:0 no greater than about 5%.

14. The process of claim 1, wherein said catalyst does not contain copper chromite.

15. A hydrogenated composition prepared by the process of claim 1.

16. A lubricant comprising a hydrogenated fatty acyl-containing composition having a ratio of monounsaturated content to saturated content of greater than about 2.

17. The lubricant of claim 16, wherein the fatty acyl containing-composition is a hydrogenated vegetable oil.

18. The lubricant of claim 17, wherein the hydrogenated vegetable oil is comprised of fatty acid chains having a profile selected from the group consisting of:
(a) C18:1/C18:0 above about 6.0; C18:2/C18:0 above about 6.0; and
C18:0 no greater than about 5%;
(b) C18:1/C18:0 above about 10.0; C18:2/C18:0 above about 3.2; and
having C18:0 no greater than about 5%;
(c) C18:1/C18:0 above about 13.9; C18:2/C18:0 above about 3.9; and
having C18:0 no greater than about 5%; and
(d) C18:1/C18:0 above about 10.7; C18:2/C18:0 above about 7.2; and
having C18:0 no greater than about 5%.

19. A method of producing a lubricant, comprising:
contacting a first composition containing at least two sites of unsaturation with a hydrogenation catalyst comprising basic copper carbonate in a hydrogen atmosphere under conditions effective to form a hydrogenated composition,
said hydrogenated composition having a ratio of monounsaturated content to saturated content of greater than about 2.

20. The method of claim 19, wherein said first composition is a polyunsaturated vegetable oil and wherein the hydrogenated composition has a saturated content that is not substantially higher than the saturated content of said first composition.

21. (canceled)
